(19) United States
(12) Patent Application Publication
(10) Pub. No.: US 2021/0402936 A1
(43) Pub. Date: Dec. 30, 2021

(54) AUTOMOBILE INTERIOR SANITIZATION

(71) Applicant: GM Global Technology Operations LLC, Detroit, MI (US)

(72) Inventors: Brooks S. Mann, Royal Oak, MI (US); Margarita M. Mann, Royal Oak, MI (US); Holly C. Schwarzwalder, Hazel Park, MI (US); Nicholas R. Estes, Hazel Park, MI (US)

(21) Appl. No.: 16/912,046

(22) Filed: Jun. 25, 2020

(51) Int. Cl. B60R 15/00; B60Q 3/20; B60R 25/01; B60R 25/31; A61L 2/24; A61L 2/10

(52) U.S. Cl. CPC B60R 15/00; B60Q 3/20; B60R 25/01; B60R 25/31; A61L 2202/11; A61L 2/10; A61L 2202/25; A61L 2202/14; A61L 2/24

(57) ABSTRACT

An automobile, comprising an interior compartment, a sanitization system adapted to sanitize surfaces within the interior compartment, a controller adapted to activate the sanitization system when at least one of a plurality of operating conditions exists and when there are no passengers present within the interior compartment, and to lock all doors to the interior compartment prior to activating the sanitization system, the sanitization system including a plurality of ultraviolet light arrays, each ultraviolet light array comprising a plurality of ultraviolet light fixtures, wherein each of the plurality of ultraviolet light fixtures is an ultraviolet light emitting diode adapted to emit ultraviolet light having a wavelength between approximately 200 nanometers and approximately 280 nanometers, at least one of the plurality of ultraviolet light arrays adapted to articulate when the sanitization system is active to re-direct the ultraviolet light being emitted to targeted areas within the interior compartment.

### INTRODUCTION

The present disclosure relates to a sanitization system for an automobile that eliminates bacterial and viral microorganisms within the passenger compartment of the automobile.

With the onset of global health issues, and a growing interest in the use of rideshare vehicles, it is important that occupants of an automobile are not exposed to bacteria or viruses that may be left behind by previous occupants. Automobiles, particularly rideshare automobiles, may see many different occupants in a day or week, and current technology is limited to the use of manual cleaning and disinfecting techniques which are time consuming and less than consistently effective.

Thus, there is a need for a new and improved system and method of sanitizing the passenger compartment of an automobile that provides frequent, on-demand sanitization of the passenger compartment that is effective at eliminating nearly all bacterial and viral microorganisms and does not take the vehicle out of service for extended periods of time.

### SUMMARY

According to several aspects of the present disclosure, an automobile includes a passenger compartment, seating components positioned within the passenger compartment, and a sanitization system positioned within the passenger compartment and adapted to sanitize surfaces within the passenger compartment and surfaces of the seating components within the passenger compartment.

According to another aspect, the sanitization system includes at least one ultraviolet light fixture adapted to project ultraviolet light onto surfaces within the passenger compartment and surfaces of the seating components within the passenger compartment.

According to another aspect, the automobile further includes a controller in communication with sensors within the automobile and adapted to activate the sanitization system when at least one of a plurality of operating conditions exists and when there are no passengers within the passenger compartment.

According to another aspect, the sanitization system is adapted to activate when the automobile has completed a trip, and when there are no passengers within the passenger compartment.

According to another aspect, the automobile is one of a hybrid automobile and an electric automobile, and the sanitization system is adapted to activate when the automobile is being charged, and when there are no passengers within the passenger compartment.

According to another aspect, the sanitization system is adapted to activate when a passenger has requested sanitization prior to using the automobile, and when there are no passengers within the passenger compartment.

According to another aspect, the sanitization system is adapted to activate when the automobile is in a maintenance mode, and when there are no passengers within the passenger compartment.

According to another aspect, the automobile is adapted to lock all doors to the passenger compartment prior to activating the sanitization system to ensure no passengers enter the automobile while the sanitization system is active.

According to another aspect, the sanitization system includes a plurality of ultraviolet light arrays, each ultraviolet light array comprising a plurality of ultraviolet light fixtures.

According to another aspect, each of the plurality of ultraviolet light fixtures within each of the plurality of ultraviolet light arrays is an ultraviolet light emitting diode adapted to emit ultraviolet light having a wavelength that is between approximately 200 nanometers and approximately 280 nanometers.

According to another aspect, at least one of the plurality of ultraviolet light arrays is adapted to articulate when the sanitization system is active to re-direct the ultraviolet light being emitted to multiple targeted areas within the interior of the passenger compartment.

According to several aspects of the present disclosure, an automobile includes an interior compartment, a sanitization system positioned within the interior compartment and adapted to sanitize surfaces within the interior compartment, and a controller in communication with sensors within the automobile and adapted to activate the sanitization system when at least one of a plurality of operating conditions exists and when there are no passengers present within the interior compartment, and to lock all doors to the interior compartment and close all windows prior to activating the sanitization system to ensure no passengers enter the interior compartment while the sanitization system is active and to ensure no ultraviolet light leaves the interior of the automobile while the sanitization system is active, the sanitization system including a plurality of ultraviolet light arrays, each ultraviolet light array comprising a plurality of ultraviolet light fixtures, wherein each of the plurality of ultraviolet light fixtures is an ultraviolet light emitting diode adapted to emit ultraviolet light having a wavelength that is between approximately 200 nanometers and approximately 280 nanometers, and at least one of the plurality of ultraviolet light arrays adapted to articulate when the sanitization system is active to re-direct the ultraviolet light being emitted to multiple targeted areas within the interior compartment.

According to several aspects of the present disclosure, a method of sanitizing surfaces within a passenger compartment and surfaces of seating components within the passenger compartment of an automobile includes activating a sanitization system when at least one of a plurality of operating conditions exists and when there are no passengers within the passenger compartment, wherein the sanitization system is mounted within the interior of the passenger compartment and includes at least one ultraviolet light fixture adapted to project ultraviolet light onto surfaces within the passenger compartment and surfaces of the seating components within the passenger compartment.

According to another aspect, the method further includes activating the sanitization system when the automobile has completed a trip, and when there are no passengers within the passenger compartment.

According to another aspect, the automobile is one of a hybrid automobile and an electric automobile, the method further including activating the sanitization system when the automobile is being charged, and when there are no passengers within the passenger compartment.

According to another aspect, the method further includes activating the sanitization system when a passenger has requested sanitization prior to using the automobile, and when there are no passengers within the passenger compartment.

According to another aspect, the method further includes activating the sanitization system when the automobile is in a maintenance mode, and when there are no passengers within the passenger compartment.

According to another aspect, the method further includes locking all doors to the passenger compartment of the automobile and closing all windows prior to activating the ultraviolet light fixtures to ensure no passengers enter the passenger compartment while the sanitization system is active and to ensure no ultraviolet light leaves the interior of the automobile while the sanitization system is active.

According to another aspect, the sanitization system includes a plurality of ultraviolet light arrays, each ultraviolet light array comprising a plurality of ultraviolet light emitting diodes adapted to emit ultraviolet light having a wavelength that is between approximately 200 nanometers and approximately 280 nanometers, and at least one of the plurality of ultraviolet light arrays is adapted to articulate when the sanitization system is active, further wherein, activating the sanitization system includes articulating the at least one of the plurality of ultraviolet light arrays to re-direct the ultraviolet light being emitted to multiple targeted areas within the interior of the passenger compartment.

According to another aspect, the automobile is a rideshare vehicle accessible by customers through an on-line application, the method further including designating the automobile as unavailable when the sanitization system is active, and designating the automobile as available when the sanitization system is no longer active.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1 is a perspective view of a passenger compartment for an automobile having seating components positioned therein and a sanitization system; and

FIG. 2 is a flow chart illustrating a method of controlling a sanitization system within an automobile.

### DETAILED DESCRIPTION

The following description is merely exemplary in nature and is not intended to limit the present disclosure, application, or uses.

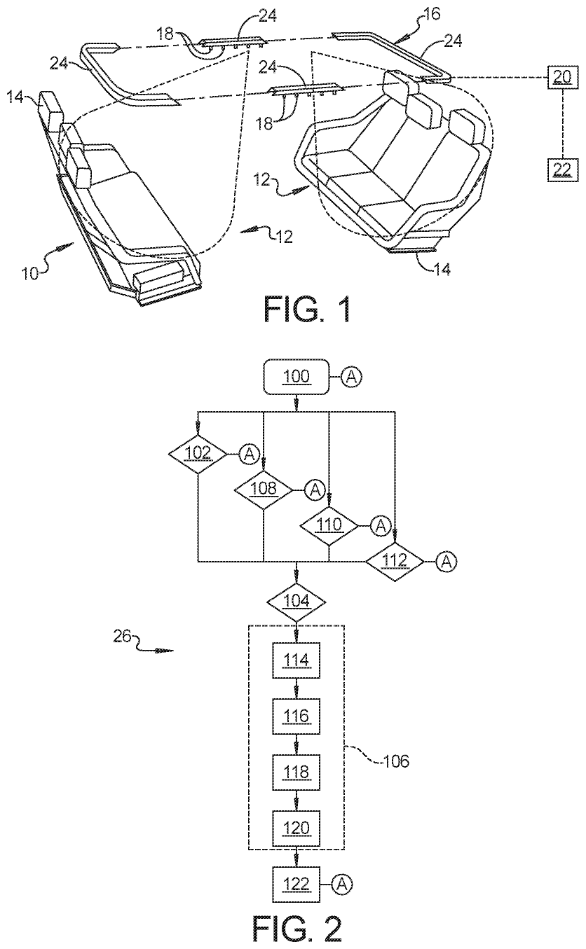

Referring to FIG. 1, a passenger compartment 12 for an automobile 10 includes seating components 14 positioned within the passenger compartment 12, and a sanitization system 16 positioned within the passenger compartment 12. The seating components 14 may be any suitable seating for an automobile 10. In the exemplary embodiment shown in FIG. 1, two rows of seats are positioned facing one another. It should be understood that any suitable arrangement of seating components 14 within the passenger compartment 12 could be incorporated without departing from the scope of the present disclosure.

The sanitization system 16 is adapted to sanitize surfaces within the passenger compartment 12 and surfaces of the seating components 14 within the passenger compartment 12. The sanitization system 16 includes at least one ultraviolet light fixture 18 that is adapted to project ultraviolet light onto surfaces within the passenger compartment 12 and surfaces of the seating components 14 within the passenger compartment 12.

It should be understood that the sanitization system 16 according to the present disclosure could be used to sanitize any interior compartment of an automobile 10. The following discussion focuses on the use of a sanitization system 16 in the passenger compartment 12 of an automobile 10, however, the sanitization system 16 could be used in an interior compartment of an automobile, such as a trunk or boot, or any other interior compartment without departing from the scope of the present disclosure.

Exposing surfaces within the passenger compartment 12 of the automobile 10 can provide greater than 99% elimination of bacteria and viruses that may be on such surfaces. If the automobile 10 is a rideshare vehicle different passengers will be using the vehicle. To provide sterilization of the passenger compartment 12 it is useful to activate the sanitization system 16 between uses. It is also important that passengers are not exposed to ultraviolet light emitted by the sanitization system 16.

In an exemplary embodiment, the automobile 10 includes a controller 20 that controls the sanitization system 16 and is in communication with devices 22 such as sensors and cameras throughout the automobile 10. The controller 20 is adapted to activate the sanitization system 16 when at least one of a plurality of operating conditions exists and when there are no passengers within the passenger compartment 12.

The controller 20 is a non-generalized, electronic control device having a preprogrammed digital computer or processor, memory or non-transitory computer readable medium used to store data such as control logic, software applications, instructions, computer code, data, lookup tables, etc., and a transceiver or input/output ports. Computer readable medium includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device. Computer code includes any type of program code, including source code, object code, and executable code.

The operating conditions that trigger activation of the sanitization system 16 are devised to trigger activation of the sanitization system 16 between uses of the automobile 10. For example, in an exemplary embodiment, the controller 20 will activate the sanitization system 16 whenever the automobile 10 has just completed a trip, and when there are no passengers within the passenger compartment 12. In another exemplary embodiment, the automobile 10 is either a hybrid or electric automobile, and the controller 20 will activate the sanitization system 16 when the automobile 10 is being charged, and when there are no passengers within the passenger compartment 12. In still another exemplary embodiment, the controller 20 will activate the sanitization system 16 when a passenger has requested sanitization prior to using the automobile 10, and when there are no passengers within the passenger compartment 12. In still another exemplary embodiment, the controller 20 will activate the sanitization system 16 when the automobile 10 is in a maintenance mode, and when there are no passengers within the passenger compartment 12. It should be understood that the controller 20 may be adapted to activate the sanitization system 16 upon the occurrence of any suitable operating condition, so long as there are no passengers within the passenger compartment 12.

Ultraviolet light can have negative effects on human passengers and pets if exposed directly to skin or eyes. Therefore, in all circumstances, the controller 20 will not activate the sanitization system 16 unless the passenger compartment 12 is empty of all living occupants. The controller 20 is in communication with devices 22 such as sensors and cameras with object detection capability within the automobile 10 to ensure that the sanitization system 16 does not activate while the passenger compartment 12 is occupied. Cameras look for the presence of occupants, such as people and pets. Weight sensors within the seats detect if an occupant is seated within the passenger compartment 12. Sensors detect if seatbelts have been buckled. These as well as other conditions are monitored to ensure that no occupants remain within the passenger compartment 12 before the sanitization system 16 will be activated.

In addition, to ensure that passengers do not enter the automobile 10 while the sanitization system 16 is active, the controller 20 will send a signal locking all doors to the passenger compartment 12 of the automobile 10. The doors to the passenger compartment 12 remain locked while the sanitization system 16 is active. When the sanitization system 16 completes a cycle, and is no longer active, the controller 20 will send a signal to unlock the doors to the passenger compartment 12. Additionally, the system will verify that all windows of the automobile are closed. This will ensure that ultraviolet light from the passenger compartment of the automobile does not leak out of the vehicle. This will prevent exposure to anyone that may be walking past or standing next to the automobile while the sanitization system is active. Further, the windows may be tinted or conditioned to block ultraviolet light from passing through the windows.

Referring again to FIG. 1, in an exemplary embodiment, the sanitization system 16 includes a plurality of ultraviolet light arrays 24. Each ultraviolet light array 24 comprises a plurality of ultraviolet light fixtures 18. To provide proper elimination of bacteria and viruses, the ultraviolet light must be germicidal short-wave ultraviolet light known as UV-C ultraviolet light. The ultraviolet light fixtures 18 within the ultraviolet light arrays 24 shown in FIG. 1 are ultraviolet light emitting diodes that are adapted to emit ultraviolet light having a wavelength that is between approximately 200 nanometers and approximately 280 nanometers. In an exemplary embodiment, the ultraviolet light fixtures 18 emit ultraviolet light having a wavelength that is approximately 253.7 nanometers.

Ultraviolet light intensity is measured in terms of wattage over an area, or micro-watts per square centimeter (μW/cm²). Ultraviolet dosage is the intensity over a period of time (μW·s/cm²). Ultraviolet intensity is generally specified for each ultraviolet light fixture 18 at a distance of 1 meter. Ultraviolet intensity rapidly increases at distances less than 1 meter.

The ultraviolet dosage required to eliminate 90% of most bacteria and viruses ranges from 2,000 to 8,000 μW·s/cm². To get closer to 99%, a dosage of 20,000 μW·s/cm² would be appropriate. By way of a non-limiting example, assume the total surface area within the passenger compartment is approximately 8 square meters (80000 cm²). 20,000 μW·s/cm²*80000 cm²=1600 Ws. If the total wattage of the ultraviolet light fixtures 18 of the sanitization system 16 is 100 watts, then the appropriate cycle time when the sanitization system 16 is activated would be approximately 16 seconds.

Effectiveness of ultraviolet germicidal irradiation depends on line-of-sight exposure of bacterial and viral microorganisms to the ultraviolet light. In an exemplary embodiment, at least one of the plurality of ultraviolet light arrays 24 is adapted to articulate when the sanitization system 16 is active to re-direct the ultraviolet light being emitted during the active cycle of the sanitization system 16 to multiple targeted areas within the interior of the passenger compartment. This helps to ensure that ultraviolet light is applied to all surfaces within the passenger compartment 12. At least one of the ultraviolet light fixtures 18 or arrays 24 may be articulating, or all of the ultraviolet light arrays 24 may be articulating. The sanitization system 16 can be designed with articulating and fixed ultraviolet light fixtures 18 and arrays 24 to ensure that all the surfaces within the passenger compartment 12 receive the proper dosage of line-of-sight ultraviolet light.

Referring to FIG. 2, a method 26 of sanitizing surfaces within a passenger compartment 12 and surfaces of seating components 14 within the passenger compartment 12 of an automobile 10 includes activating a sanitization system 16 when at least one of a plurality of operating conditions exists and when there are no passengers within the passenger compartment 12, wherein the sanitization system 16 is mounted within the interior of the passenger compartment 12 and includes at least one ultraviolet light fixture 18 adapted to project ultraviolet light onto surfaces within the passenger compartment 12 and surfaces of the seating components 14 within the passenger compartment 12.

In an exemplary embodiment, the automobile 10 includes a controller 20 that controls the sanitization system 16 and is in communication with devices 22 such as sensors and cameras throughout the automobile 10. The controller 20 is adapted to activate the sanitization system 16 when at least one of a plurality of operating conditions exists and when there are no passengers within the passenger compartment 12. The operating conditions that trigger activation of the sanitization system 16 are devised to trigger activation of the sanitization system 16 between uses of the automobile 10.

Starting at block 100, and moving to block 102, the controller 20 monitors the automobile 10 and systems within the automobile 10 to see if the automobile 10 has just completed a trip. If the automobile 10 has just completed a trip, then moving on to block 104, the controller 20 accesses devices 22 such as sensors and cameras within the automobile 10 to see if there are any passengers within the passenger compartment 12 of the automobile 10. If the automobile 10 has just completed a trip, and if the controller 20 verifies that there are no passengers within the passenger compartment 12, the controller 20 will activate the sanitization system 16, as indicted at block 106.

Simultaneously, starting at block 100, and moving to block 108, the controller 20 monitors the automobile 10 and systems within the automobile 10 to see if the automobile 10 is being charged. This operating condition is relevant if the automobile 10 is either a hybrid or electric vehicle. If the automobile 10 is being charged, then moving on to block 104, the controller 20 accesses devices 22 such as sensors and cameras within the automobile 10 to see if there are any passengers within the passenger compartment 12 of the automobile 10. If the automobile 10 is being charged, and if the controller 20 verifies that there are no passengers within the passenger compartment 12, the controller 20 will activate the sanitization system 16, as indicted at block 106.

Simultaneously, starting at block 100, and moving to block 110, the controller 20 monitors the automobile 10 and systems within the automobile 10 to see if a passenger has requested sanitization prior to using the automobile 10. If a passenger has requested sanitization prior to using the automobile 10, then moving on to block 104, the controller 20 accesses devices 22 such as sensors and cameras within the automobile 10 to see if there are any passengers within the passenger compartment 12 of the automobile 10. If a passenger has requested sanitization prior to using the automobile 10, and if the controller 20 verifies that there are no passengers within the passenger compartment 12, the controller 20 will activate the sanitization system 16, as indicted at block 106.

Simultaneously, starting at block 100, and moving to block 112, the controller 20 monitors the automobile 10 and systems within the automobile 10 to see if the automobile 10 is in a maintenance mode. If the automobile 10 is in a maintenance mode, then moving on to block 104, the controller 20 accesses devices 22 such as sensors and cameras within the automobile 10 to see if there are any passengers within the passenger compartment 12 of the automobile 10. If the automobile 10 is in a maintenance mode, and if the controller 20 verifies that there are no passengers within the passenger compartment 12, the controller 20 will activate the sanitization system 16, as indicted at block 106.

The system checks being performed by the controller 20 at blocks 102, 108, 110 and 112 are done simultaneously. If the result of the checks performed at any one of blocks 102, 108, 110 and 112 are positive, then the controller 20 moves on to block 104 to verify that there are no passengers (human or pets) within the passenger compartment 12. If the results of the checks performed at all of the blocks 102, 108, 110 and 112 are negative, then the controller 20 reverts back to block 100. The controller 20 will continue to perform system checks at blocks 102, 108, 110 and 112 on a periodic cycle, for example, once every set time period, or anytime the automobile 10 is shut off or started.

When any one of the plurality of operating conditions is present and when there are no passengers within the automobile 10, the controller 20 will activate the sanitization system 16, as indicated by block 106. Activation of the sanitization system 16 includes multiple steps. Starting at block 114, the controller 20 locks all of the doors to the passenger compartment 12 within the automobile 10 to ensure no passengers enter the passenger compartment 12 while the sanitization system 16 is active. Additionally, the controller 20 verifies that all the windows in the automobile 10 are closed, to prevent leakage of ultraviolet light from the automobile 10 while the sanitization system in active.

Moving to block 116, the automobile 10 may be a ride-share vehicle that customers access through an on-line application. If the automobile 10 is a ride-share vehicle, the controller 20 sends a signal to the on-line application indicating that the automobile 10 is not available while the sanitization system 16 is active.

Moving to block 118, the controller 20 activates the ultraviolet light fixtures 18 of the sanitization system 16. The sanitization system 16 includes a plurality of ultraviolet light arrays 24, each ultraviolet light array 24 comprising a plurality of ultraviolet light emitting diodes adapted to emit ultraviolet light having a wavelength that is between approximately 200 nanometers and approximately 280 nanometers. While the sanitization system 16 is active, the controller 20 articulates at least one of the plurality of ultraviolet light arrays 24 to re-direct the ultraviolet light being emitted to multiple targeted areas within the interior of the passenger compartment 12.

Moving to block 120, once the interior of the automobile 10 has been exposed to a sufficient dosage of ultraviolet light, the controller 20 de-activates the sanitization system 16. Finally, Moving to block 122, after the sanitization system 16 has been de-activated, the controller 20 unlocks the doors to the passenger compartment 12, and if appropriate, sends notification to the on-line application that the automobile 10 is once again available as a ride-share vehicle.

After de-actuation of the sanitization system 16 the controller 20 once again returns to block 100, as indicated by connector blocks A, where the process of checking for appropriate operating conditions once again begins.

An automobile 10 having a sanitization system 16 of the present disclosure offers several advantages. These include providing a sterilized passenger compartment to different passengers that may utilize the automobile 10. The sanitization is automatic and takes a very short time, thereby providing very little interference to usage of the automobile 10.

The description of the present disclosure is merely exemplary in nature and variations that do not depart from the gist of the present disclosure are intended to be within the scope of the present disclosure. Such variations are not to be regarded as a departure from the spirit and scope of the present disclosure.

What is claimed is:

1. An automobile, comprising:
a passenger compartment;
seating components positioned within the passenger compartment; and
a sanitization system positioned within the passenger compartment and adapted to sanitize surfaces within the passenger compartment and surfaces of the seating components within the passenger compartment.

2. The automobile of claim 1, wherein the sanitization system includes at least one ultraviolet light fixture adapted to project ultraviolet light onto surfaces within the passenger compartment and surfaces of the seating components within the passenger compartment.

3. The automobile of claim 2, further including a controller in communication with sensors within the automobile and adapted to activate the sanitization system when at least one of a plurality of operating conditions exists and when there are no passengers within the passenger compartment.

4. The automobile of claim 3, wherein the sanitization system is adapted to activate when the automobile has completed a trip, and when there are no passengers within the passenger compartment.

5. The automobile of claim 3, wherein the automobile is one of a hybrid automobile and an electric automobile, and the sanitization system is adapted to activate when the automobile is being charged, and when there are no passengers within the passenger compartment.

6. The automobile of claim 3, wherein the sanitization system is adapted to activate when a passenger has requested sanitization prior to using the automobile, and when there are no passengers within the passenger compartment.

7. The automobile of claim 3, wherein the sanitization system is adapted to activate when the automobile is in a maintenance mode, and when there are no passengers within the passenger compartment.

8. The automobile of claim 3, wherein the automobile is adapted to lock all doors to the passenger compartment and to verify that all windows are closed prior to activating the sanitization system to ensure no passengers enter the automobile while the sanitization system is active and to ensure no ultraviolet light leaves the interior of the automobile while the sanitization system is active.

9. The automobile of claim 8, wherein the sanitization system includes a plurality of ultraviolet light arrays, each ultraviolet light array comprising a plurality of ultraviolet light fixtures.

10. The automobile of claim 9, wherein each of the plurality of ultraviolet light fixtures within each of the plurality of ultraviolet light arrays is an ultraviolet light emitting diode adapted to emit ultraviolet light having a wavelength that is between approximately 200 nanometers and approximately 280 nanometers.

11. The automobile of claim 10, wherein at least one of the plurality of ultraviolet light arrays is adapted to articulate when the sanitization system is active to re-direct the ultraviolet light being emitted to multiple targeted areas within the interior of the passenger compartment.

12. An automobile, comprising:
an interior compartment;
a sanitization system positioned within the interior compartment and adapted to sanitize surfaces within the interior compartment;
a controller in communication with sensors within the automobile and adapted to activate the sanitization system when at least one of a plurality of operating conditions exists and when there are no passengers present within the interior compartment, and to lock all doors to the interior compartment prior to activating the sanitization system to ensure no passengers enter the interior compartment while the sanitization system is active;
the sanitization system including a plurality of ultraviolet light arrays, each ultraviolet light array comprising a plurality of ultraviolet light fixtures, wherein each of the plurality of ultraviolet light fixtures is an ultraviolet light emitting diode adapted to emit ultraviolet light having a wavelength that is between approximately 200 nanometers and approximately 280 nanometers;
at least one of the plurality of ultraviolet light arrays adapted to articulate when the sanitization system is active to re-direct the ultraviolet light being emitted to multiple targeted areas within the interior compartment.

13. A method of sanitizing surfaces within a passenger compartment and surfaces of seating components within the passenger compartment of an automobile comprising:
activating a sanitization system when at least one of a plurality of operating conditions exists and when there are no passengers within the passenger compartment, wherein the sanitization system is mounted within the interior of the passenger compartment and includes at least one ultraviolet light fixture adapted to project ultraviolet light onto surfaces within the passenger compartment and surfaces of the seating components within the passenger compartment.

14. The method of claim 13, further including activating the sanitization system when the automobile has completed a trip, and when there are no passengers within the passenger compartment.

15. The method of claim 13, wherein the automobile is one of a hybrid automobile and an electric automobile, the method further including activating the sanitization system when the automobile is being charged, and when there are no passengers within the passenger compartment.

16. The method of claim 13, further including activating the sanitization system when a passenger has requested sanitization prior to using the automobile, and when there are no passengers within the passenger compartment.

17. The method of claim 13, further including activating the sanitization system when the automobile is in a maintenance mode, and when there are no passengers within the passenger compartment.

18. The method of claim 13, further including locking all doors to the passenger compartment of the automobile and verifying that all windows are closed prior to activating the ultraviolet light fixtures to ensure no passengers enter the passenger compartment while the sanitization system is active and to ensure no ultraviolet light leaves the interior of the automobile while the sanitization system is active.

19. The method of claim 18, wherein the sanitization system includes a plurality of ultraviolet light arrays, each ultraviolet light array comprising a plurality of ultraviolet light emitting diodes adapted to emit ultraviolet light having a wavelength that is between approximately 200 nanometers and approximately 280 nanometers, and at least one of the plurality of ultraviolet light arrays is adapted to articulate when the sanitization system is active, further wherein, activating the sanitization system includes articulating the at least one of the plurality of ultraviolet light arrays to re-direct the ultraviolet light being emitted to multiple targeted areas within the interior of the passenger compartment.

20. The method of claim 19, wherein the automobile is a rideshare vehicle accessible by customers through an on-line application, the method further including:
designating the automobile as unavailable when the sanitization system is active; and
designating the automobile as available when the sanitization system is no longer active.
